(19) United States
(12) Patent Application Publication
(10) Pub. No.: US 2025/0146872 A1
(43) Pub. Date: May 8, 2025

(54) SYSTEM AND METHOD FOR TEMPERATURE PROFILING WITH RAMAN SCATTERING

(71) Applicant: Michigan Aerospace Corporation, Ypsilanti, MI (US)

(72) Inventor: William Eric JOHNSON, Saline, MI (US)

(21) Appl. No.: 18/838,601

(22) PCT Filed: Feb. 14, 2023
(86) PCT No.: PCT/US2023/062540

§ 371 Date: Aug. 14, 2024

Related U.S. Application Data
(60) Provisional application No. 63/309,679, filed on Feb. 14, 2022.

(51) Int. Cl. G01J 3/44; G01K 11/125; G01N 33/18

(52) U.S. Cl. CPC G01J 3/44; G01K 11/125; G01N 33/18

(57) ABSTRACT

The invention is directed to a system and method for temperature profiling based on using Raman scattering spectral shape changes that occur with temperature and the concentration of one or more components. In the case of water, as an example, Raman scattering spectral shape changes that occur with temperature and salinity are used. Raman scattering from liquid or solid water can be used to provide a temperature profile as a function of water depth/range without requiring contacting the water or air containing the water. A Raman spectrum can be analyzed to determine the molecule in the water that is responsible for the spectrum. Raman scattering is an inelastic process where the Raman scattered photons have a different frequency than the incident photon. The amount of the frequency shift depends upon the characteristics of the scattering medium. The invention is able to observe the Raman scattering from multiple ranges simultaneously.

### I. BACKGROUND OF INVENTION

### Scope of the Invention

Raman scattering from fluid mediums (i.e., liquid or gas), whether the medium is in liquid gaseous or even solid form, such as liquid or solid water, can be used to provide a temperature profile as a function of the depth of the fluid medium without requiring contacting the fluid medium. The invention described herein is able to observe the Raman scattering from multiple depths simultaneously.

### Summary of the Prior Art

Thermometers, thermistors, and thermocouples have all been used to measure the temperature of water, as an example, but they must contact the water to make a temperature measurement. Non-contact Infrared systems can measure water temperature without contacting the water by measuring the infrared light emitted from the water's surface. However, infrared measurements are limited to the surface because the of the high absorption of water at the infrared wavelengths. Measurements at the near infrared wavelengths are not practical as the black body radiation spectrum drops off so rapidly that there is not enough signal to make reliable measurements. Contact and non-contact temperature measurement systems also lack the ability to simultaneously measure other environmental parameters of interest such as phase state of the fluid or concentrations of other fluids or gases of interest.

### II. SUMMARY OF THE INVENTION

This invention is based on knowing how the Raman scattering spectral shape changes with temperature and the concentration of components in the fluid medium. In particular, using water as an example, the invention is based on Raman scattering spectral shape changing with temperature and salinity. Data on the salinity of the water of interest (i.e., freshwater or saltwater) is provided by the user. Raman scattering is an inelastic process where the Raman scattered photons have a different frequency than the incident photon. The amount of the frequency shift depends upon the characteristics of the scattering medium. A Raman spectrum can be analyzed to determine the molecule that is responsible for the spectrum.

Instruments that utilize Raman scattering typically operate at shorter wavelengths when compared to the infrared sensors due to an increase in Raman scattering efficiency with shorter incident wavelengths. In an embodiment used for measuring water, as an example, the laser wavelength is 405 nm and is close to the peak water transmission allowing one to see further into the water. In this embodiment, the laser beam and receiving optics are arranged so that the object plane is angled so that it is in focus along the image plane, as was first described by Scheimpflug and as is well known in the art of optics.

### III. BRIEF DESCRIPTION OF THE DRAWINGS

The present invention is illustrated in the accompanying drawings, wherein:

FIG. 1 illustrates how the Raman spectrum shape changes as the temperature changes;

FIG. 2 shows a sensor schematic;

FIG. 3 is A block diagram showing the operations of an active range resolved Raman spectroscopy;

FIG. 4A a cross section of the receiver optics consisting of a field lens, entrance slit, collimator, grating, focusing lens, and camera sensor;

FIG. 4B is a block diagram showing how the collected optical signal is processed into a hyperspectral image;

FIG. 5A shows how the elastic scatter a single line and Raman scatter broader area;

FIG. 5B is an image where the dark indicates more signal while the lighter areas have less signal; and

FIG. 5C is an example where the spectroscopy can capture the Raman spectra in addition to the fluorescent or absorption spectra.

### IV. DESCRIPTION OF THE PREFERRED EMBODIMENT

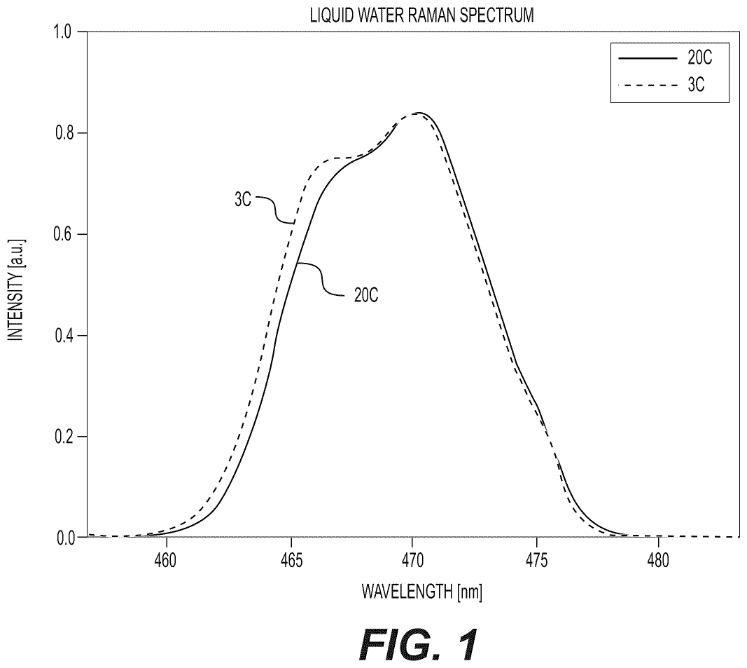

The embodiments of the present invention will be described hereinbelow in conjunction with the above-described drawings. In addition, the embodiments will be described using water as the example fluid medium, though other fluid mediums, whether they be in their liquid, gas or solid forms, are equally applicable to the present invention. Accordingly using water as the example, the change in the liquid water Raman spectrum for temperatures of 20° C. and 3° C. with illumination by a laser emitting at 405 nm is shown in FIG. 1. In calculating the Raman spectral shape, the water's salinity or freshness must be included in the mathematical characterization of the Raman spectrum so as to fit to a specific temperature uniquely.

The shape of the spectrum can indicate the phase of the water and if the air temperature is known can indicate if the liquid water is in a supercritical state. As the temperature changes the water spectrum, which is typically considered a superposition of multiple emission peaks of unequal intensities, transitions from a spectrum where emission peaks corresponding to water in a monomer form at higher liquid temperatures to a spectrum where the emission peak originating from water molecules in a polymer solid form e.g. ice. Water vapor Raman emission occurs at shorter wavelengths near the liquid/solid water Raman spectrum. Thus by measurement of the spectrum the user can match the spectrum to that of water of a specific phase and temperature.

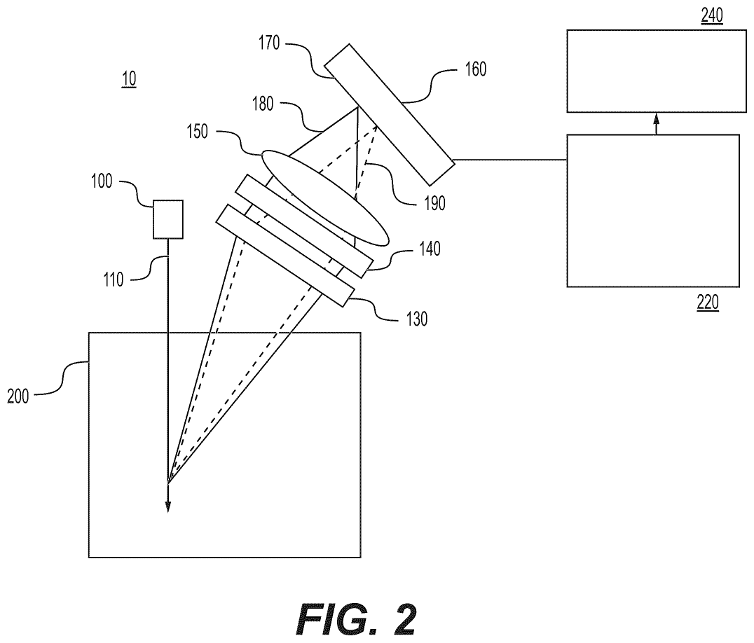

A schematic of the Raman Temperature Sensor 10 according to the present invention is shown in FIG. 2. The light source is a laser 100 that outputs a nearly collimated laser beam 110. The laser beam 110 illuminates the water 200 where most of the light is transmitted, wherein some of the laser beam is scattered by the water 200. In the scattered laser beam light, changes in the energy of the photons occur at different levels; some photon energies do not change even after scattering. Thus, there are two components of photons with different energy levels in the scattered light 120. Specifically, the light scattered by the Raman effect produces photons that are both higher in energy and lower in energy. The lower energy photons are referred to as Stokes and the higher energy photons are referred to as anti-Stokes. The Raman Temperature Sensor 10 according to the present invention uses the Stokes component as it is stronger than the anti-Stokes.

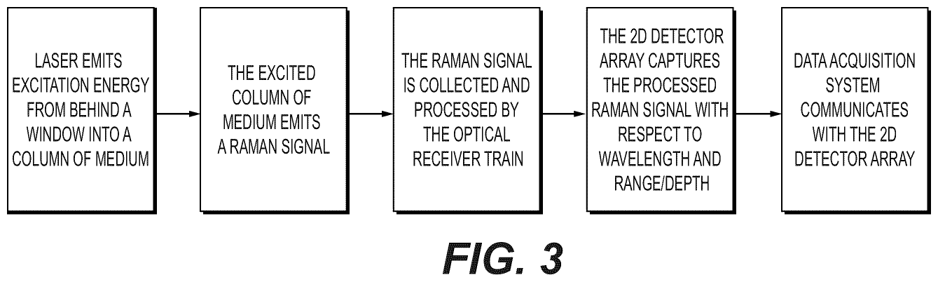

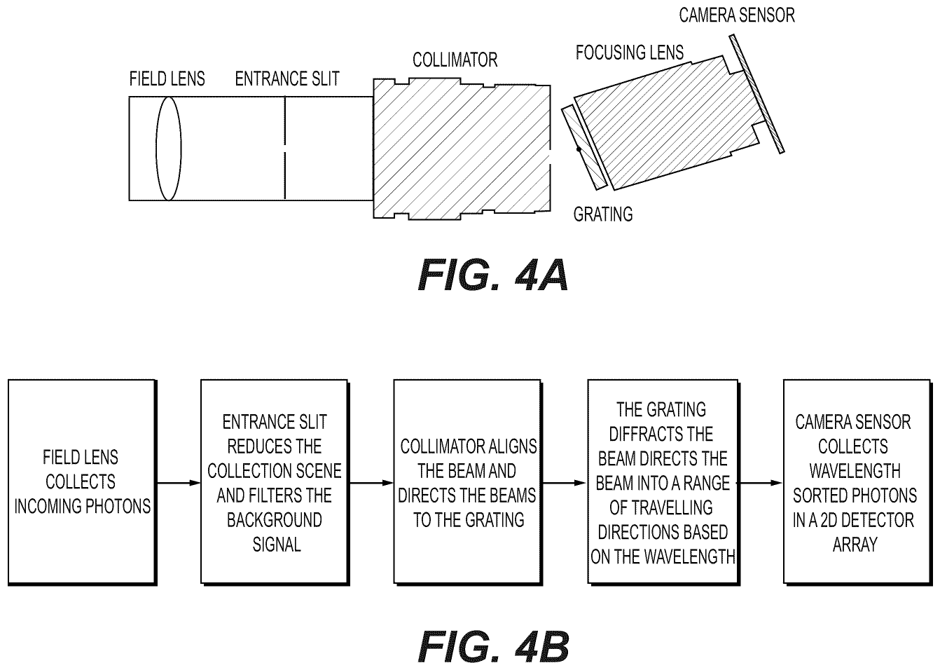

In operation, the Raman Temperature Sensor 10 collects data on multiple Raman spectra resulting from light scattered from different depths simultaneously, while the light from the laser 100 is directed through the volume of the water 200. As illustrated in FIG. 2, the light scattered from a point along the laser beam 110 (i.e., at a particular depth) travels through and out of the water, and then encounters a bandpass filter 130 of the Raman Temperature Sensor 10 that is used to reduce the background illumination. The filtered scattered light then goes through a dispersing element 140 where the light is dispersed to produce a Raman spectrum. The lens 150 focuses the dispersed light onto an image plane 170. A more detailed diagram describing the principle of operation of the sensor and the receiver collection optics are shown in FIGS. 3 and 4, respectively.

A detector array 160, such as a camera or other similar optical device, is physically offset from the light source and positioned to be able to view a length of the focused light that passes through the lens 150 on the image plane 170. The focused light that reaches the image plane 170 is elastic scattering 180 that is imaged through the dispersing element 140, such as a transmission grating, which disperses the light orthogonal to the direction of light beam propagation before being focused by the lens 150. As represented in FIG. 2, the elastic scatter 180 represents the scattered laser energy detected by the Raman Temperature Sensor 10 that does not undergo any change in the energy of the scattered photons.

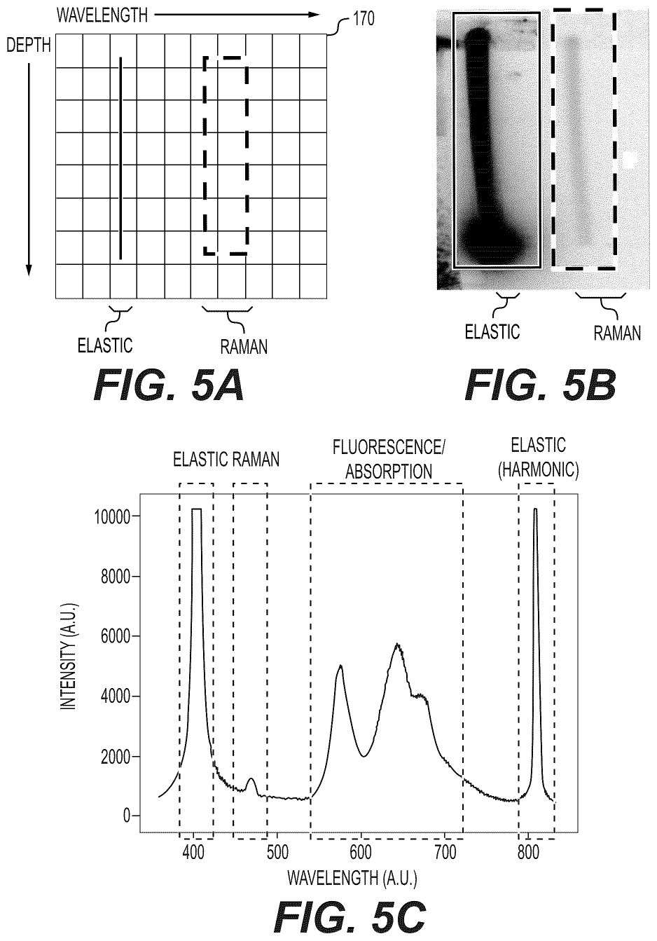

FIG. 5A shows how the elastic scattering 180 that reaches the image plane 170 from the dispersing element 140 would be graphically translated by a processing circuit 220 connected to receive data signals from the image plane 170 and then shown on a display 240. Specifically, the signal from elastic scattering 180 resulting from the particular depth and light scattering through the water that is detected on the image plane 170 produces an image of the beam in the water column in one area of the detector array 160. That image is represented by the heavy line on the left of FIG. 3A. The box outlined by the dashed line shows how the Raman emission 190 appears separated from the elastic scattering 180 on the detector array image plane 170. Each row in the image pertains to a unique distance from the camera, which enables the Raman emission 190 to be analyzed independently for each depth in the field of view and the corresponding temperature calculated.

FIG. 5B shows an actual image of the elastic scatter 180 and the Raman scatter 190. In this image, the intensity is represented in the reverse, wherein a brighter section appears darker, and the lower signal level is lighter. The elastic scatter 180 appears to be wider than the Raman scatter 190 image because it is so much stronger than the Raman signal that it appears to be wider. However, the spectrum of the laser is much narrower than the Raman scatter bandwidth.

FIG. 5C shows when configured to span a larger spectral range than that corresponding to water alone, the instrument can measure the emission of other Raman and fluorescence wavelengths for the quantification of other trace gas or liquid components mixed in the volume being measured. These spectral features can be measured simultaneously with the water measurements provided the spectral range of the instrument spans those emission features corresponding to a gas of interest such as oxygen.

Although the present invention has been fully described in connection with the preferred embodiment thereof with reference to the accompanying drawings, it is to be noted that various changes and modifications will be apparent to those skilled in the art. Such changes and modifications are to be understood as included within the scope of the present invention as defined by the appended claims unless they depart therefrom.

I/we claim:

1. A system for determining vibrational Raman spectra of water at multiple depths, comprising:
a collimated light source configured to illuminate a body of water, wherein collimated light from the light source is at least partially scattered by the water so as to generate scattered light directed out of the body of water;
a light receiving element configured to collect the scattered light directed out of the body of water;
at least one dispersing element configured to disperse the collected scattered light to generate at least a Raman spectrum;
at least one detector array configured to receive and detect at least the Raman spectrum resulting from the dispersed scattered light; and
a processing circuit operatively connected to the detector array and configured at least to generate at least a temperature profile with respect to depth of the body of water in response to the Raman spectrum resulting from the dispersed scattered light, wherein the scattered light directed out of the body of water is scattered in response to characteristics of the body of water or air including at least one of depth, temperature, water salinity, water freshness, water phase, and other substances with emissions that can be detected within the spectral range of the instrument.

2. A system according to claim 1, further comprising:
a bandpass filter configured to receive the scattered light and to reduce the background illumination therefrom.

3. A system according to claim 1, further comprising:
an image plane configured to receive the Raman spectrum resulting from the dispersed scattered light generated by the dispersing element, wherein the detector element is operatively connected to receive the dispersed scattered light imaged on the image plane.

4. A system according to claim 3, further comprising:
an optical lens assembly configured to receive and focus the dispersed light from the dispersing element onto the image plane.

5. A system according to claim 1, wherein the processing circuit includes a display operatively connected and configured to at least generate a graphical representation of the temperature profile with respect to depth of the body of water in response to the Raman.

6. A method for remote sensing of water temperature at different ranges by analysis of the Raman spectra emitted by different ranges, comprising the steps of:
providing a Raman spectra detecting device including at least a collimated light source configured to illuminate a body of water, a dispersing element configured to disperse scattered light, and a detector array;
directing the collimated light source at the body of water so as to generate light scattered by and directed out of the body of water;
receiving and dispersing the scattered light in the dispersing element to generate a Raman spectrum;
detecting the Raman spectrum resulting from the dispersed scattered light;
generating at least a temperature profile of the body of water in response to the Raman spectrum resulting from the dispersed scattered light, wherein the scattered light directed out of the body of water is scattered in response to characteristics of the body of water including at least one of depth, temperature, water salinity, water freshness, water phase, and other constituent concentrations.

7. A method according to claim 6, wherein the step of generating at least a temperature profile of the body of water includes determining temperatures at the different ranges by simultaneous measurement of Raman spectra from each depth in the water column.

8. A method according to claim 6, wherein the step of generating at least a temperature profile of the body of water includes determining temperatures at the different ranges by simultaneous measurement of Raman spectra and incorporating supplemental information from external sources.

9. A method according to claim 6, wherein the step of generating at least a temperature profile of the body of water or trace amounts of water in air includes determining temperatures at the different ranges by a form of analytical or numerical or machine learning solutions.

10. A system for determining vibrational Raman spectra of a fluid medium at multiple depths, comprising:
a collimated light source configured to illuminate a body of fluid medium, wherein collimated light from the light source is at least partially scattered by the fluid medium so as to generate scattered light directed out of the body of fluid medium;
a light receiving element configured to collect the scattered light directed out of the body of fluid medium;
at least one dispersing element configured to disperse the collected scattered light to generate at least a Raman spectrum;
at least one detector array configured to receive and detect at least the Raman spectrum resulting from the dispersed scattered light; and
a processing circuit operatively connected to the detector array and configured at least to generate at least a temperature profile with respect to depth of the body of fluid medium in response to the Raman spectrum resulting from the dispersed scattered light, wherein the scattered light directed out of the body of fluid medium is scattered in response to characteristics of the body of fluid medium or air including at least one of depth, temperature, concentration of a component in the fluid medium, fluid medium freshness, fluid medium phase, and other substances with emissions that can be detected within the spectral range of the instrument.

11. A system according to claim 10, further comprising:
a bandpass filter configured to receive the scattered light and to reduce the background illumination therefrom.

12. A system according to claim 10, further comprising:
an image plane configured to receive the Raman spectrum resulting from the dispersed scattered light generated by the dispersing element, wherein the detector element is operatively connected to receive the dispersed scattered light imaged on the image plane.

13. A system according to claim 12, further comprising:
an optical lens assembly configured to receive and focus the dispersed light from the dispersing element onto the image plane.

14. A system according to claim 10, wherein the processing circuit includes a display operatively connected and configured to at least generate a graphical representation of the temperature profile with respect to depth of the body of fluid medium in response to the Raman.

15. A method for remote sensing of a temperature of a fluid medium at different ranges by analysis of the Raman spectra emitted by different ranges, comprising the steps of:
providing a Raman spectra detecting device including at least a collimated light source configured to illuminate a body of fluid medium, a dispersing element configured to disperse scattered light, and a detector array;
directing the collimated light source at the body of fluid medium so as to generate light scattered by and directed out of the body of fluid medium;
receiving and dispersing the scattered light in the dispersing element to generate a Raman spectrum;
detecting the Raman spectrum resulting from the dispersed scattered light;
generating at least a temperature profile of the body of fluid medium in response to the Raman spectrum resulting from the dispersed scattered light, wherein the scattered light directed out of the body of fluid medium is scattered in response to characteristics of the body of fluid medium including at least one of depth, temperature, concentration of a component in the fluid medium, fluid medium freshness, fluid medium phase, and other constituent concentrations.

16. A method according to claim 15, wherein the step of generating at least a temperature profile of the body of fluid medium includes determining temperatures at the different ranges by simultaneous measurement of Raman spectra from each depth in the fluid medium column.

17. A method according to claim 15, wherein the step of generating at least a temperature profile of the body of fluid medium includes determining temperatures at the different ranges by simultaneous measurement of Raman spectra and incorporating supplemental information from external sources.

18. A method according to claim 15, wherein the step of generating at least a temperature profile of the body of fluid medium or trace amounts of fluid medium in air includes determining temperatures at the different ranges by a form of analytical or numerical or machine learning solutions.
